(19) United States
(12) Patent Application Publication
(10) Pub. No.: US 2020/0375469 A1
(43) Pub. Date: Dec. 3, 2020

(54) Methods of Monitoring White Spots on Teeth

(71) Applicant: Colgate-Palmolive Company, New York, NY (US)

(72) Inventors: LaTonya KILPATRICK-LIVERMAN, Princeton, NJ (US); Jianhong QIU, Warren, NJ (US); Ralph Peter SANTARPIA, III, Edison, NJ (US)

(73) Assignee: Colgate-Palmolive Company, New York, NY (US)

(21) Appl. No.: 16/606,785

(22) PCT Filed: Apr. 24, 2018
(86) PCT No.: PCT/US2018/029134

§ 371 Date: Oct. 21, 2019

Related U.S. Application Data
(60) Provisional application No. 62/489,366, filed on Apr. 24, 2017.

(51) Int. Cl. A61B 5/00; G06T 7/00

(52) U.S. Cl. CPC A61B 5/0088; G06T 7/0012; G06T 2207/10024; G06T 2207/30096; G06T 2207/30036

(57) ABSTRACT

The disclosure provides methods of quantifying the severity or monitoring the progression of white spot lesions on a tooth by quantifying the grayscale difference between the white spot lesions and the surrounding healthy enamel in the blue channel of a digital color photographic image of the tooth.

### BACKGROUND

Dental enamel is a thin, hard layer of calcified material that covers the crown of teeth. The major mineral component of dental enamel is hydroxyapatite, a crystalline form of calcium phosphate. Poorly mineralized regions beneath, within or adjacent to intact enamel often appear as white spots on the teeth because such poorly mineralized regions are porous and reflect light differently from enamel. In most cases, white spots are the result of demineralization, for example from acids produced by cariogenic bacteria, which break down the hydroxyapatite into calcium and phosphate ions, leading to porous regions within the enamel. Other common causes of poor mineralization and associated white spot lesions include xerostomia, trauma, and arrested decay that has only partially remineralized around fixed orthodontic appliances, which may provide shelter for bacteria or interfere with normal remineralization. Conditions that interfere with tooth development, such as fluorosis, hypomineralization/hypomaturation, mineral deficiencies, and hypoplasia, can also cause white spot lesions.

The diagnosis and evaluation of white spots is typically performed simply by looking at the teeth. The development of the white spots and/or the efficacy of treatments to remove white spots typically judged simply by looking at “before” and “after” pictures. Such evaluation is subjective and qualitative. The results depend to a large extent on the person making the assessment and the lighting conditions when the assessment is made.

It is possible to measure and compare grayscale values of teeth in a photograph, but the contrast between the white spot and the surrounding white enamel using conventional grayscale values is generally inadequate to provide a degree of distinction sufficient to measure fine changes or differences in the white spots and is subject to distortion by the lighting conditions.

There is a need for improved methods of quantifying the severity of white spot lesions on the teeth, to allow more accurate assessment and monitoring of the white spots.

### BRIEF SUMMARY

The present inventors have developed a new method of quantifying the severity of white spot lesions on the teeth.

Color digital images are made of pixels, a pixel being the smallest controllable element of a digital picture. Pixels in a color digital image are made of combinations of primary colors represented by a series of code. Grayscale is a measure of intensity. A “channel” in this context is the grayscale image of the same subject as a color image, made of just one of these primary colors. For instance, an image from a standard digital camera will have a red, green and blue channel. CMYK images for printing have four channels for cyan, magenta, yellow and black ink. The digital code for each pixel therefore provides an address (where in the image it is located) and a grayscale (intensity value) for each channel. The blend of the intensity of each channel in each pixel and the blend of pixels in the picture all combine to provide the image that we see.

This disclosure provides methods of quantifying the severity and monitoring the progression of white spot lesions on a tooth by quantifying the grayscale difference between the white spot lesions and the surrounding healthy enamel in the blue channel of a digital color photographic image of the tooth. Focusing on the contrast in the blue spectrum highlights the differences between the white spot areas and the sound enamel (which is naturally slightly yellow and therefore absorbs blue light) and digitally filters out much of the variability generated by different lighting in different environments, providing a more accurate and consistent measure of the severity of the white spot relative to the adjacent enamel. The blue channel is the channel corresponding to a color with a wavelength in the range of 450-520 nm, e.g., a color corresponding to about 450-495 nm, e.g. about 470 nm.

For example, in one embodiment, the disclosure provides a novel method for quantifying the severity of white spots on teeth, comprising
a) drying the tooth to be evaluated;
b) obtaining a color photographic image of the tooth surface in digital form (e.g. in digital form comprising red, green and blue channel information); and
d) measuring the difference between the grayscale value of at least one pixel in the area depicting at least a portion of the white spot (e.g. of the pixel having high or the highest grayscale value in such area) and the grayscale value of at least one pixel in the area depicting at least a portion of the adjacent healthy enamel (e.g. of the pixel having low or the lowest grayscale value in such area).

The difference between the the grayscale value of the pixel in the area depicting the white spot and the grayscale value of the pixel in the area depicting the adjacent healthy enamel is a measure of the contrast between the white spot and the new adjacent enamel. The greater the contrast, the more noticeable and/or severe the white spot is. If the contrast increases over time, that indicates the white spot is getting worse. If the contrast reduces, that indicates the severity of the white spot is reduced, at least from an aesthetic perspective.

In another embodiment the disclosure provides a method of evaluating a treatment for white spot on the teeth of a patient comprising quantifying the severity of white spots on teeth as described above before and after such treatment, wherein a reduction in the difference between the grayscale value of at least one pixel in the area depicting at least a portion of the white spot and the grayscale value of at least one pixel in the area depicting at least a portion of the adjacent healthy enamel indicates the efficacy of such treatment.

For example, the white spot repair efficacy of a proposed treatment can be calculated as follows:
1) Before treatment (baseline): Contrast 1=blue channel grayvalue of white spot (highest value)—blue channel grayvalue of adjacent area of sound enamel (lowest value);
2) After treatment: Contrast 2=blue channel grayvalue of white spot (highest value) blue channel greyvalue of adjacent area of sound enamel (lowest value) (Same areas are measured for baseline and post treatment);
3) White spot repair efficacy of the treatment=Contrast 2−Contrast 1
   Negative values represent decreased contrast, corresponding to white spot lesion repair that includes improved aesthetics, e.g., the white spot color is closer to the color of the surrounding enamel and therefore less visible.

In another embodiment the disclosure provides a method of monitoring the progress of a white spot condition on the teeth of a patient comprising quantifying the severity of white spots on teeth over time, as described above, wherein a reduction in the difference between the grayscale value of at least one pixel in the area depicting at least a portion of the white spot and the grayscale value of at least one pixel in the area depicting at least a portion of the adjacent healthy enamel indicates repair of the white spot.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

Digital cameras and image processing software are widely available, and virtually all image processing programs permit shifting of color balance, which would enable them to be used for the methods of the instant disclosure.

For example, ImageJ is a public domain, Java-based image processing program developed at the National Institutes of Health, which can be downloaded free at https://imagej.nih.gov/ij/. Using ImageJ, the operations can be carried out as follows:
1. Take baseline and post treatment images on white spot and adjacent sound area (same area(s) for baseline and post-treatment) using SpectroShade.
2. Open the image files being compared at once so that they all can be seen on the screen together.
3. Click on the window of interest (e.g baseline at first)
4. Click Image->Color>Split Channels, and it will split the image into three channels—green, red, and blue.
5. The blue channel shows the best binary image. Delete the other two.
6. Select an area of the baseline image to be analyzed. Use the rectangle tool to drag a rectangle across the desired area it should contain part of the white spot and part of the normal crown of the tooth. The purpose of this is to compare the contrast of the white spot versus the normal part, a.k.a. how white is the white spot compared to the rest of the tooth?
4. After the area is chosen, retrieve the minimum and maximum values (darkest and lightest):
   4-a. Click Analyze □ Plot Profile and a window will pop up with a plot of the contrast by pixel.
   4-b. Click the List button to see a list of values
   4-c. Go through the list, record the lowest and highest values.
5. Calculate the difference between the minimum and maximum and record. This difference is the Contrast 1 (between white spot and sound area).
6. Repeat for the post-treatment image (step 3-5). Make sure same area is analyzed for baseline and post-treatment. Then Contrast 2 is obtained from post-treatment image.
7. The contrast differences between baseline and post-treatment (contrast 2 contrast 1) reflect the effect of the treatment. More negative (less contract compared to baseline) means better white spot lesion repair (removal).

The following examples illustrate methods for use in accordance with the present disclosure. The exemplified methods are illustrative and do not limit the scope of the disclosure.

### EXAMPLES

### Example 1—Measuring White Spot Reduction

Human extracted teeth with white spot(s) are gently brush cleaned and also screened by dentist. The screened teeth are then stored in deionized water at ca. 4° C. until the treatments are started.

The stored teeth are taken out, slightly dried with paper towel before the base line images of the white spots and adjacent sound areas are taken. The contrast values between the white spot and adjacent sound areas are quantified using ImageJ.

The teeth are then soaked in one of three test solutions or control mouthwash for 30 minutes, 15 ml for each tooth, at 37° C., while being spun at 100 rpm. The teeth are then rinsed for 5 minutes with deionized water at 400 rpm. The teeth are then paper towel dried, and the deionized water rinse is repeated. The treated teeth are then incubated in artificial saliva overnight at 37° C.

This treatment, washing and incubation is repeated daily for nine more days, for a total of 10 days of treatment. After the 10 days of treatment is completed, the teeth are rinsed twice with deionized water as described.

In order to avoid false repair readings, e.g., decreased contrast from loosely deposited mineral (from artificial saliva) on tooth surface, the teeth are brushed using medium toothbrush after all treatment/incubation. The teeth are each brushed under tap water, continuously and evenly at all angles for two minutes, rinsed under tap water, and placed onto a paper towel to dry.

Final images of the treated teeth, after brushing, are taken after the teeth are slightly dried with the paper towel. The contrast between the white spot area and the adjacent sound enamel is again quantified. Reduction in contrast (negative value) indicates degree of repair of the white spot. The results are set forth in Table 2:

TABLE 2

| Material | Contrast change |
|---|---|
| 1 | −21.4 |
| 2 | −18.6 |
| 3 | −19.5 |
| Control | 0.76 |

This data shows that the three novel treatment solutions reduced the white spot lesions, while the control treatment had little effect on the white spots.

While particular embodiments of the invention have been illustrated and described, it will be obvious to those skilled in the art that various changes and modifications may be made without departing from the scope of the invention as defined in the appended claims.

1. A method of quantifying the severity or monitoring the progression of white spot lesions on a tooth comprising quantifying the grayscale difference between the white spot lesions and the surrounding healthy enamel in the blue channel of a digital color photographic image of the tooth.

2. The method of claim 1, comprising
a) drying the tooth to be evaluated;
b) obtaining a digital color photographic image of the tooth; and
d) measuring the difference between the grayscale value of at least one pixel in an area of the image depicting at least a portion of the white spot and the grayscale value of at least one pixel in an area of the image depicting at least a portion of the adjacent healthy enamel, wherein the grayscale values are measured only in the blue channel of the image.

3. The method of claim 1 wherein the digital color photographic image of the tooth surface in digital form comprises red, green and blue channel information.

4. The method of claim 1 wherein the blue channel of the digital color photographic image is a channel that provides a color having a wavelength of in the range of 450-520 nm.

5. The method of claim 2 wherein the at least one pixel in the area depicting at least a portion of the white spot is a pixel having high grayscale value in such area, and the least one pixel in the area depicting at least a portion of the adjacent healthy enamel is a pixel having low grayscale value in such area.

6. The method of claim 2 wherein the amount of difference between the grayscale value of the pixel in the area depicting the white spot and the grayscale value of the pixel in the area depicting the adjacent healthy enamel corresponds to the severity of the white spot.

7. A method of evaluating a treatment for white spot on the teeth of a patient comprising quantifying the severity of white spots on teeth in accordance with claim 1 before and after such treatment, wherein a reduction in the difference between the grayscale value of at least one pixel in the area depicting at least a portion of the white spot and the grayscale value of at least one pixel in the area depicting at least a portion of the adjacent healthy enamel indicates the efficacy of such treatment.

8. A method of monitoring the progress of a white spot condition on the teeth of a patient comprising quantifying the severity of white spots on teeth over time, in accordance with claim 1, wherein a reduction in the difference between the grayscale value of at least one pixel in the area depicting at least a portion of the white spot and the grayscale value of at least one pixel in the area depicting at least a portion of the adjacent healthy enamel indicates repair of the white spot.
